(19) United States
(12) Patent Application Publication
(10) Pub. No.: US 2025/0017476 A1
(43) Pub. Date: Jan. 16, 2025

(54) ACTIVE IMPLANTABLE SENSOR

(71) Applicant: SYNKOPI, INC., Palo Alto, CA (US)

(72) Inventor: Alan OSTROFF, Pleasanton, CA (US)

(21) Appl. No.: 18/714,250

(22) PCT Filed: Nov. 29, 2022
(86) PCT No.: PCT/US2022/080595

§ 371 Date: May 29, 2024

Related U.S. Application Data
(60) Provisional application No. 63/283,654, filed on Nov. 29, 2021.

(51) Int. Cl. A61B 5/021; A61B 5/00

(52) U.S. Cl. CPC A61B 5/021; A61B 5/686; A61B 2560/0209; A61B 2560/0214; A61B 2560/0462; A61B 2562/0223; A61B 2562/0247; A61B 2562/028; A61B 2562/168

(57) ABSTRACT

The present disclosure is directed to systems and methods for measuring pressure within a patient. In some implementations, an active implantable sensor is provided that is configured to continuously or periodically obtain pressure signal measurements from within a heart of a patient. The AIS can have an operational lifetime up to or exceeding 5-7 years, and can be configured to continuously or periodically transmit recorded information or data or statistics synthesized from the recorded information to an external device, such as a smartphone, tablet, pc, or other electronic device. Methods of use are also provided.

### RELATED APPLICATIONS

This application claims priority to U.S. Prov. Ser. No. 63/283,654 filed on Nov. 29, 2021, titled “Active Implantable Sensor” which is incorporated herein by reference in its entirety.

### INCORPORATION BY REFERENCE

All publications and patent applications mentioned in this specification are herein incorporated by reference to the same extent as if each individual publication or patent application was specifically and individually indicated to be incorporated by reference.

### FIELD

The present disclosure details novel systems and methods for measuring pressure in a patient with an active implantable sensor. Specifically, this disclosure provides an active implantable sensor for continuously measuring pressure in the heart of the patient over extended operational lifetimes (e.g., up to 5-7 years or more) without requiring an external charging device or battery replacement.

### BACKGROUND

Implantable cardiac pressure monitors are used to monitor changes in heart pressure (e.g., pulmonary artery pressure) which can provide early indications of worsening heart failure.

Previously disclosed implantable pressure monitors, for example CardioMEMs, Endotronix, and Vectorious Medical Technologies, are not active devices; they require a wand or other device to be placed close to an implanted sensor in order to communicate with the passive implant to measure intracardiac pressure. The drawback from using a passive implant is that the sensor can only measure and record information when the wand is in close proximity since there is no internal power source or memory elements in the implant to record activity.

The CardioMEMs and Endotronix sensor both measure PAP (pulmonary artery pressure). Vectorious Medical measures LAP (left atrial pressure) by fixating their sensor in the atrial septal wall.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 is a schematic drawing showing electronics of an active implantable sensor.

FIG. 2 is one embodiment of an active implantable sensor including a deformable lid, a pressure sensor, an electronics compartment, and a cell.

FIGS. 3-4 illustrate examples of an active implantable sensor with an antenna.

FIGS. 5A-5C illustrate one embodiment of an active implantable sensor having a hermetic cavity for housing the sensor.

FIGS. 6, 7A-7B, and 8 illustrate examples of electronics and/or an antenna on a substrate and housed within the electronics compartment of an active implantable sensor.

FIGS. 9A-9C illustrate one embodiment of implanting an active implantable sensor in a heart of a patient.

### SUMMARY OF THE DISCLOSURE

An active implantable pressure sensor is provided, comprising a housing adapted to be implanted within a human heart, a cell disposed in the housing, a pressure sensor disposed in the housing and configured to measure pressure information outside of the housing, a transmitter disposed on or in the housing and configured to transmit the pressure information, a supply holding capacitor electrically connected to the cell, the supply holding capacitor being configured to provide current to the transmitter during transmission, and a microprocessor configured to control transmission of the pressure information by the transmitter, wherein the microprocessor is configured to intermittently turn on the transmitter for a transmit-time sufficient to avoid a supply voltage of the active implantable pressure sensor from collapsing and turn off the transmitter for an off-time sufficient to allow the cell to recharge the supply holding capacitor above a recharge threshold.

Another active implantable pressure sensor is provided, comprising a housing adapted to be implanted within a human heart, a cell disposed in the housing, a pressure sensor disposed in the housing and configured to measure pressure information outside of the housing, a receiver disposed on or in the housing and configured to receive information from an external device, a supply holding capacitor electrically connected to the cell, the supply holding capacitor being configured to provide current to the receiver during receiving, and a microprocessor configured to control receiving on information from the external device, wherein the microprocessor is configured to intermittently turn on the receiver for a receive-time sufficient to avoid a supply voltage of the active implantable pressure sensor from collapsing and turn off the receiver for an off-time sufficient to allow the cell to recharge the supply holding capacitor above a recharge threshold.

An active implantable pressure sensor, comprising a housing adapted to be implanted within a human heart, a cell disposed in the housing, a pressure sensor disposed in the housing and configured to measure pressure information outside of the housing, a transceiver disposed on or in the housing and configured to transmit the pressure information and receive information from an external device, a supply holding capacitor electrically connected to the cell, the supply holding capacitor being configured to provide current to the transceiver during transmit and receive, and a microprocessor configured to control transmission of the pressure information by the transmitter, wherein the microprocessor is configured to intermittently turn on the transceiver for an on-time sufficient to avoid a supply voltage of the active implantable pressure sensor from collapsing and turn off the transceiver for an off-time sufficient to allow the cell to recharge the supply holding capacitor above a recharge threshold

A pressure measurement system is provided, comprising an external device, and an active implantable sensor including a housing adapted to be implanted within a human heart of a patient, a cell disposed in the housing, a pressure sensor disposed in the housing and configured to measure pressure information outside of the housing, a transmitter, receiver, or transceiver disposed on or in the housing and configured to transmit the pressure information to the external device and receive information from the external device, a supply holding capacitor electrically connected to the cell, the supply holding capacitor being configured to provide current to the transmitter, receiver, or transceiver during transmit and receive, and a microprocessor configured to control transmission of the pressure information by the transmitter, wherein the microprocessor is configured to intermittently turn on the transceiver for an on-time sufficient to avoid a supply voltage of the active implantable sensor from collapsing and turn off the transceiver for an off-time sufficient to allow the cell to recharge the supply holding capacitor above a recharge threshold.

In any of the sensors or systems described above or herein, the sensor and/or system may further include the following embodiments:

In some embodiments, the housing comprises a separate hermetically sealed cavity for housing the pressure sensor. In some embodiments, the separate hermetically sealed cavity is oil filled. In one example, the separate hermetically sealed cavity comprises a deformable lid. In some embodiments, the separate hermetically sealed cavity further comprises an oil fill port that is sealed with a ball and lid.

In some implementations, the pressure sensor comprises a MEMs capacitive sensor.

In one example, the housing comprises a cell compartment for housing the cell, an electronics compartment for housing the transmitter and microprocessor, and a hermetically sealed cavity for housing the pressure sensor. In some embodiments, the electronics compartment comprises a volume of between 0.25 and 0.75 cc.

In some embodiments, the sensor or system includes an antenna operatively coupled to the transmitter. In one implementation, the antenna is integrated into the housing. In some examples, the antenna is hermetically sealed within the housing.

In one example, the transmitter and microprocessor are disposed on a substrate. In another implementation, the substrate comprises a flex substrate, wherein the flex substrate is folded or bent to fit in the housing.

In some implementations of the sensor or system, an antenna is disposed on the flex substrate. In one embodiment, the antenna is folded or wrapped around at least a portion of the transmitter or microprocessor. In some examples, the substrate comprises a first section and a second section attached with a connecting portion of substrate, wherein the substrate is configured to bend or fold on the connecting portion, wherein the transmitter and microprocessor are disposed on the first section and second section. In another implementation, the substrate further comprises a third section attached to the second section with a second connecting portion of substrate, wherein the third section of substrate includes an antenna.

In some embodiments, the sensor is configured to have an operating lifetime of at least 5 years. In on example, the operating lifetime of 5 years is based on a housing volume between 0.5 cc and 2.0 cc and a cell volume of up to 0.75 cc. In some examples, the housing has a volume of between 0.5 cc and 2.0 cc.

In some embodiments, the microprocessor is configured to synthesize and compress the pressure information before transmission.

In another embodiment, the sensor or system can comprise a giant magnetoresistance (GMR) sensor.

In some implementations, the external device is a self-contained unit. In some examples, the external device is a smart phone.

In one embodiment, the external device is configured to push alerts to the patient based on the pressure information received from the active implantable sensor.

In some examples, the external device is configured to receive an input from the patient.

A method of measuring pressure within a heart of a patient is provided, comprising the steps of delivering an active implantable sensor into the heart of the patient, measuring pressure information of the heart with a pressure sensor of the active implantable sensor, transmitting the pressure information from the active implantable sensor to an external device for a transmit-time sufficient to avoid a supply voltage of the active implantable pressure sensor from collapsing, and turning off transmission intermittently for an off-time sufficient to allow a supply holding capacitor of the active implantable pressure sensor to recharge above a recharge threshold.

In some embodiments, the method comprises performing the measuring, transmitting, and turning off steps at least once a day for at least 5 years without recharging, replacing, or externally powering the active implantable pressure sensor.

In another implementation, the method includes synthesizing and compressing the pressure information before the transmitting step.

In some examples, sending an alert to the patient with the external device.

In some examples, the method includes transmitting an acknowledgement signal from the external device to the active implantable sensor that the pressure information was received. In one embodiment, the acknowledgment signal includes a received signal strength indicator (RSSI). In one example, the method includes modifying transmit settings of the active implantable sensor based on the RSSI to reduce power during subsequent transmissions.

In some examples, the method includes attempting to transmit the pressure information until the acknowledgement signal is received.

An active implantable pressure sensor is provided, comprising a housing having a volume less than 2 cc and adapted to be implanted within a human heart, a cell disposed in the housing, a pressure sensor disposed in the housing and configured to measure a pressure reading outside of the housing, a deformable lid attached to the housing and being fluidly coupled with the pressure sensor, and electronics disposed in the housing and operatively coupled to the cell and the pressure sensor, the electronics being configured to synthesize blood pressure statistics from the pressure reading and intermittently transmit the blood pressure statistics to an external device multiple times per day over an operating lifetime of at least 5 years.

In some embodiments, the housing comprises a separate hermetically sealed cavity for housing the pressure sensor. In one embodiment, the separate hermetically sealed cavity is oil filled. In some embodiments, the deformable lid is attached to the separate hermetically sealed cavity. In one example, the separate hermetically sealed cavity further comprises an oil fill port that is sealed with a ball and lid.

In one embodiment, the pressure sensor comprises a MEMs capacitive sensor.

In some examples, the electronics comprise a real time clock, a transceiver, and a capacitive to digital converter.

In one embodiment, the electronics compartment comprises a volume of between 0.25 and 0.75 cc.

In another embodiment, the sensor includes an antenna operatively coupled to the electronics. In some examples, the antenna is integrated into the housing. In one embodiment, the antenna comprises a loop antenna.

In some examples, the electronics are disposed on a substrate. In one embodiment, the substrate comprises a flex substrate, wherein the flex substrate is folded or bent to fit the electronics in the housing. In another embodiment, an antenna is disposed on the flex substrate. In one example, the antenna is folded or wrapped around at least a portion of the electronics. In some embodiments, the substrate comprises a first section and a second section attached with a connecting portion of substrate, wherein the substrate is configured to bend or fold on the connecting portion, wherein the electronics are disposed on the first section and second section. In another embodiment, the substrate further comprises a third section attached to the second section with a second connecting portion of substrate, wherein the third section of substrate includes an antenna.

In some examples, the operating lifetime of 5 years is based on a housing volume between 0.5 cc and 2.0 cc and a cell volume of up to 0.75 cc.

A heart pressure measurement system is provided, comprising an active implantable pressure sensor adapted to be implanted in a human heart, the sensor comprising a housing having a volume less than 2 cc, a cell disposed in the housing, a pressure sensor disposed in the housing and configured to measure a pressure reading outside of the housing, a deformable lid attached to the housing and being fluidly coupled with the pressure sensor, and electronics disposed in the housing and operatively coupled to the cell and the pressure sensor, the electronics being configured to synthesize blood pressure statistics from the pressure reading and transmit the blood pressure statistics multiple times per day and receive communications over an operating lifetime of at least 5 years; and an external device in wireless communication with the active implantable sensor, the external device being configured to receive transmitted blood pressure statistics from the active implantable sensor and transmit communications to the active implantable sensor.

A method of measuring pressure within a heart of a patient, comprising delivering a fully implantable active pressure sensor into the heart of the patient, frequently or continuously obtaining one or more pressure readings of the heart per day with the sensor, synthesizing blood pressure statistics from the one or more pressure readings, transmitting the blood pressure statistics to an external device, and repeating the obtaining, synthesizing, and transmitting steps over an operating lifetime of at least 5 years without recharging or replacing a power source of the sensor.

### DETAILED DESCRIPTION

This disclosure describes an active implantable sensor (AIS) that is configured to be implanted inside the heart and to measure, store, and transmit diagnostic information (e.g., pressure measurement) via wireless communication to a nearby relay. The AIS of the present disclosure includes a power source, such as a battery cell, and is designed and configured to perform the pressure sensing and transmission of data over a prescribed implantation lifetime of 5-7 years or more, without requiring an external device for charging the cell or to activate the device or initiate data collection.

In a preferred embodiment, the AIS is delivered via a catheter to various locations inside the heart. The AIS can be cylindrically shaped, leadless, between 0.5 cc and 2 cc in volume, and can be attached to a steerable catheter for delivery inside the heart. Typically, the femoral vein is a common entry point for such a device and the AIS can be steered by catheter into the right atrium, right ventricle, pulmonary artery, or delivered transeptally to access the left atrial chamber or left ventricle. Fixation of the (AIS) is not covered in detail in this disclosure, however screws, barbs, clips, and other fasteners can be incorporated in the AIS to secure the device to the heart wall.

In a preferred embodiment, a self-powered (e.g., battery powered) AIS measures heart pressure and potentially other parameters such as an endocardial electrogram or activity throughout the day and wirelessly transmits this diagnostic data to a nearby relay. The relay could then be configured to send this information to a centrally located data center for further analysis and diffusion. The data center provides a portal for physicians and other care givers. The data center can also send data such as, instructions, messages, thresholds, etc., back to the relay and ultimately to the AIS. The relay may be placed relatively near (within a few meters) the patient and can also incorporate visual and audible outputs such as a display and speaker to alert the patient to one or more actions that should be taken. The relay could also incorporate input devices such as a touch-screen to receive input(s) from the patient, such as allowing the patient to acknowledge instructions or alerts posted to the patient. The relay could conveniently be located anywhere in the home or on person. Multiple relays could also be installed in proximity to the patient.

In some embodiments, the relay can comprise stand-alone devices such as smartphones, tablets, a pc, or other computing devices that communicate wirelessly with the AIS and connect to the cloud or a remote server.

One of the challenges of making the AIS is device longevity given the volume constraints. It's desirable for such a device to have a longevity of at least 3 years minimum and typically at least 5-7 years, since the medical procedure to implant the AIS occurs at some monetary expense and medical risk.

It is the objective of this device to make an AIS that requires no patient involvement or clumsy equipment to retrieve diagnostic information from the heart, with the exception of the patient being in proximity to a relay. This disclosure provides a self-powered AIS that records information throughout the day and transmits data wirelessly to a relay regularly.

It is an objective of this device to be powered by a primary cell so that it can perform the aforementioned functionality with a longevity of 5 years of more. Another embodiment could use a secondary cell or supercapacitor requiring infrequent re-charging. In a preferred embodiment, the AIS would send diagnostic information to the relay at least once per day.

This daily update would include information collected throughout the day, something that passive sensors and others that use a similar principle cannot achieve.

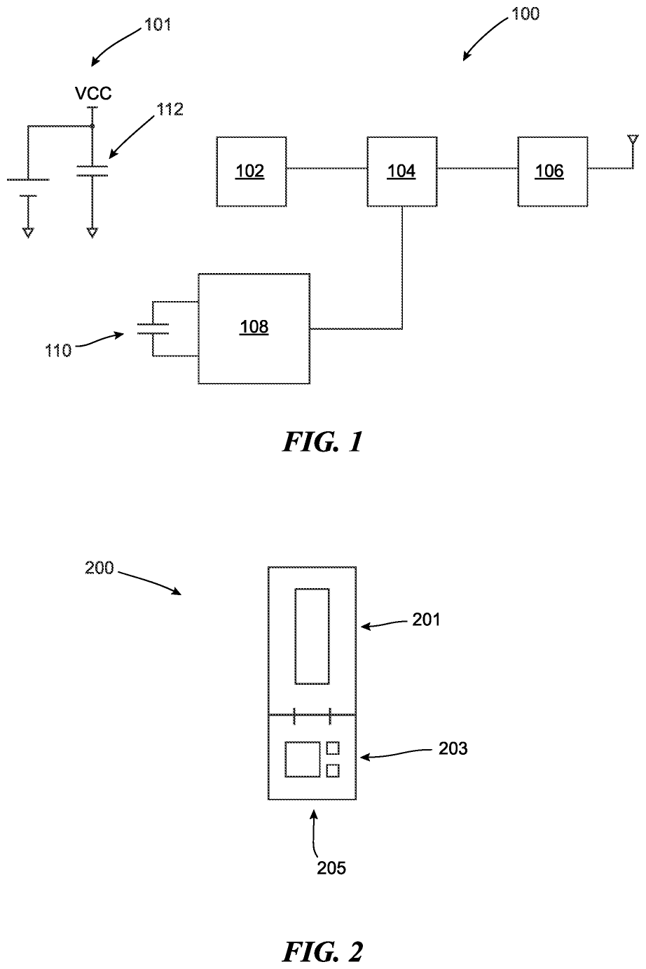

A simplified system design diagram of an AIS 100 of the present disclosure is shown in FIG. 1, which includes electronics such as a cell 101, a real-time-clock RTC 102, a microprocessor (μP) 104, a transceiver 106, and a pressure sensor/capacitive-to-digital converter 108, and pressure sensor 110. While the pressure sensor 112 is shown as a capacitive pressure sensor, it should be understood that other suitable pressure sensors can be used. Energy is provided to the AIS by the cell 101, which can include a supply holding capacitor 112 across the cell. The supply holding capacitor is configured to supply energy during the current intensive operations of the AIS such as transmit and receive of the transceiver. This is because the impedance of the cell is too large to provide current during these operations. In one specific embodiment, the holding capacitor can have a capacitance ranging from 10 uF to 200 uF. VCC is used to power the system. The external RTC 102 is used to keep the other components in their idle state. When required, the RTC will interrupt the μP 104 to either collect and process pressure sensor data, or activate the transceiver 106 to wirelessly send data to the relay. Not shown is an analog-to-digital-converter (ADC) configured to measure the cell voltage to determine a recommended replacement time. Additionally, the electronics can include memory for pressure measurement data storage. In one embodiment, the electronics can include a giant magnetoresistance (GMR) sensor to provide a simple way to aid in testing and configuration of the AIS. For example, the GMR sensor could immediately wake-up the device for communication and instructions from the relay.

In order to a provide reliable, long lasting, and efficient device, the active implantable sensor of the present disclosure can are specifically designed and configured to prevent the supply voltage of the cell/battery from collapsing during use. For any given configuration of the AIS (e.g., cell size, transceiver/microprocessor power requirements, operational lifetime, etc.), there exists a minimum supply voltage beyond which the AIS will no longer be able to function properly.

As described above, the supply holding capacitor is configured to supply energy to the AIS electronics during use, particularly during current intensive operations such as transmit/receive. The size of the holding capacitor can be specifically chosen to meet the size requirements of the device and the voltage requirements of operation. Too large of a supply holding capacitor and the capacitor takes up too much room or exhibits too much leakage current. Too small of a supply holding capacitor is insufficient to provide the needed energy. In one non-limiting implementation, the supply holding capacitor can have a capacitance ranging from approximately 10 uF to 200 uF to help prevent the supply voltage of the cell from collapsing during use.

Another factor that can prevent the supply voltage of the cell from collapsing during use is to limit the transmit on time. When the transmitter is turned on, the AIS can exhibit a large current drain on the order of many milliamps. As described above, the current for transmit comes from the supply holding capacitor due to the large cell impedance. As will be described in more detail below, if the AIS of the present disclosure were configured to transmit all pressure measurements/data at once the supply holding capacitor voltage will drop and the AIS won't have sufficient voltage to run properly. Therefore, the AIS of the present disclosure is configured to transmit in small bursts or windows (e.g., a selected transmit-on time repeated over a set time period) to avoid a supply voltage of the active implantable pressure sensor from collapsing, with sufficient time between these bursts or windows (e.g., a selected transmit-off time interspersed with the transmit-on time) for the cell to recharge the supply holding capacitor.

In some embodiments, the supply voltage collapsing is defined as when the supply voltage is too low or insufficient to support operation of the AIS. In one specific embodiment, the transmit-on times can be controlled to prevent the supply voltage from collapsing by up to 100 mV. In other embodiments, the transmit-on times can be controlled to prevent the supply voltage from collapsing by up to 200 mV, up to 300 mV, up to 500 mV, or up to 1000 mV.

The transmit-off times can be controlled to allow the cell to recharge the supply holding capacitor above a recharge threshold. In some embodiments, this recharge threshold can be at least 60%, at least 70%, at least 80%, at least 90%, at least 95%, or up to at least 99% of the supply voltage. In some embodiments, the recharge threshold is based on the current requirements of the transmitter/transceiver during transmit-on times. Therefore, the recharge threshold can be based on the current requirements and transmit-on times of the transmitter/transceiver. In some embodiments, this recharge threshold can be automatically determined and changed in response to the transmit-on/transmit-off times of the transmitter/transceiver. In one specific example, the microprocessor can be configured to determine the transmit-on times sufficient to prevent the supply voltage from collapsing, and can separately determine the transmit-off times sufficient to recharge the supply holding capacitor to supply voltage sufficient to maintain transmission during the entirety of the transmit-on times.

Similarly, the AIS of the present disclosure is configured to limit the amount of time the receiver is turned on to prevent the supply holding capacitor voltage from dropping beyond a voltage threshold and to allow sufficient time between receive-on time for the cell to recharge the supply holding capacitor.

The basic construction of an AIS 200 according to one embodiment is shown in the cutaway view of FIG. 2. Here, the AIS 200 includes a hermetically sealed cell 201 that is coupled to a hermetically sealed electronics compartment 203 with a deformable lid 205 integrating the pressure sensor. The deformable lid may contain capacitive elements that can be sensed by the capacitive-to-digital converter, or may contain resistive elements that can also be used to sense pressure. In the case of a resistive elements, the capacitive-to-digital converter would be replaced by an analog-to-digital converter than can be configured measure the change in resistance. In some embodiments, the cell can comprise a volume of up to 0.75 cc or up to 1.0 cc. The electronics compartment can occupy a volume of between 0.25 cc and 0.75 cc, or between 0.25 cc and 1.0 cc. In some embodiments, the cell and the electronics compartment are collectively disposed in a single housing or can. In other embodiments, the electronics compartment comprises a first housing that is connected, attached, or coupled to a separate housing of the cell. In general, embodiments of the AIS disclosed herein can include one or more separate components making up the housing of the device.

Any of the active implantable sensors (AIS) disclosed herein can be configured to continuously or periodically measure pressure waveforms within the heart. In some examples, the raw pressure waveforms can be transmitted to an external device (e.g., a relay, or an electronic device such as a smartphone, tablet, PC, or other external component of the pressure measurement system) and/or stored locally on the device. Alternatively, the AIS can be configured to calculate, compile, or synthesize periodic (e.g., hourly or some other user specified time period) measurements of systolic, diastolic and mean pressure, as well as heart rate statistics such as mean heart rate and variability, and transmit those synthesized measurements or statistics to the external device or relay. In some embodiments, the AIS can be configured to store and/or transmit only the last measured pressure, or last calculated statistic.

The electronics of the AIS can further be configured to extract the patient's heart rate from the measured pressure waveform. For example, in one embodiment the electronics can include an endocardial electrogram amplifier for direct EGM measurements. In some examples, ECG statistics can be stored periodically such as hourly along with a daily minimum heart rate, max heart rate, and heart rate variability. Any of the extracted or calculated data can be transmitted by the AIS to the external device.

In some examples, the AIS can send a low data, low power “ping” signal to the relay or external device when none of the calculated statistics or data hit a programmable threshold. This can help the AIS to avoid sending data transmissions when there is nothing to report, helping to increase operational life of the AIS.

The AIS can also be configured to receive communications from the external device or relay. In some embodiments, the relay is configured to send an acknowledgement signal to the AIS when a data transmission signal is received by the relay. In one example, the relay or external device can include a received signal strength indicator (RSSI) in the acknowledgement signal so the AIS can modify transmit settings accordingly in real-time to save power during transmissions.

In some embodiments, the AIS can be automatically configured to measure and store pressure and electrogram snapshots when a patient has an “event” or “alarm condition” as defined within the system. For example, an event or alarm can be defined as a pressure exceeding or falling below a defined threshold, or a heart rate reading that falls above or below a heart rate threshold. In some examples, these “events” can automatically trigger communication of the latest pressure readings and statistics to an external device. In some embodiments, the AIS could detect an alarm or event condition based on pre-set thresholds and rules that interpret the on-board patient statistics that have been collected and send an alert to the relay. The AIS could be configured to continue trying to transmit throughout the day until the alert is acknowledged by the relay. The alarm could be based on analysis of history or could occur immediately following a measurement.

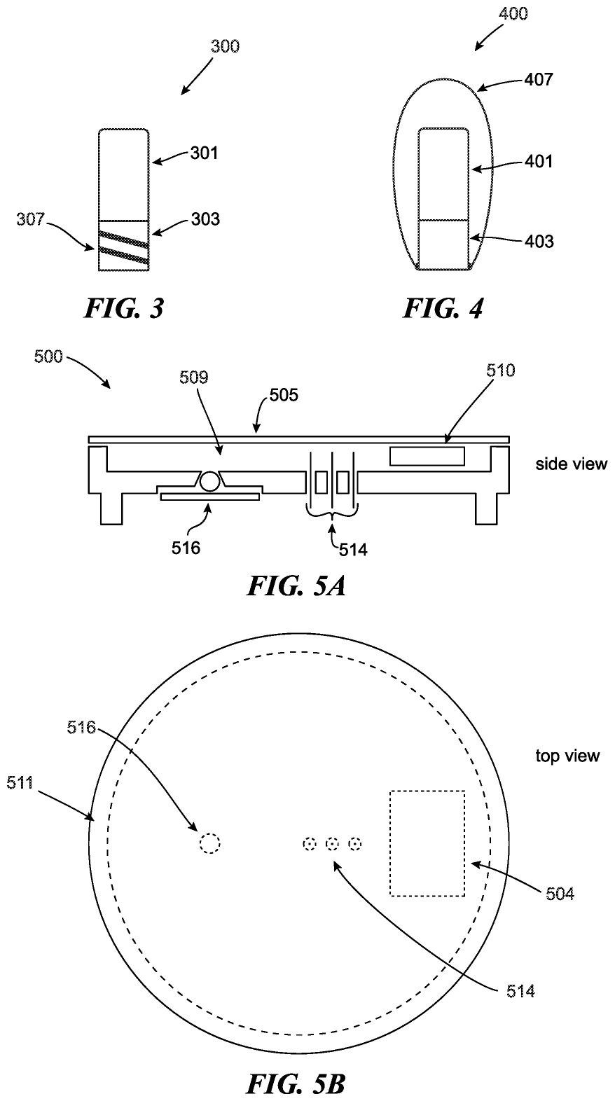

The embodiment of FIG. 2 includes but does not show an antenna for transmitting/receiving data. The antenna can comprise a Medical Implant Communications Service (MICS), Industrial, Scientific and Medical (ISM), Bluetooth or similar band antenna that can be integrated into the AIS in a number of ways. In FIG. 3, the AIS 300 can include a cell 301 and a ceramic electronics compartment 303. An antenna 307 can be integrated on the ceramic enclosure that houses the electronics. These antennas are typically loop (magnetic field) antennas and less susceptible to body impedance issues compared to electric field antennas. In the embodiment of FIG. 4, the AIS 400 can include a cell 401, titanium electronics compartment 403, and antenna 407. In this example, the antenna is implemented using an exterior nitinol loop antenna. In the embodiment of FIG. 4, one end of the loop antenna can be coupled or attached to the can, and the other end can be electronically coupled to the electronics in the electronics compartment via a hermetic feedthrough in the can.

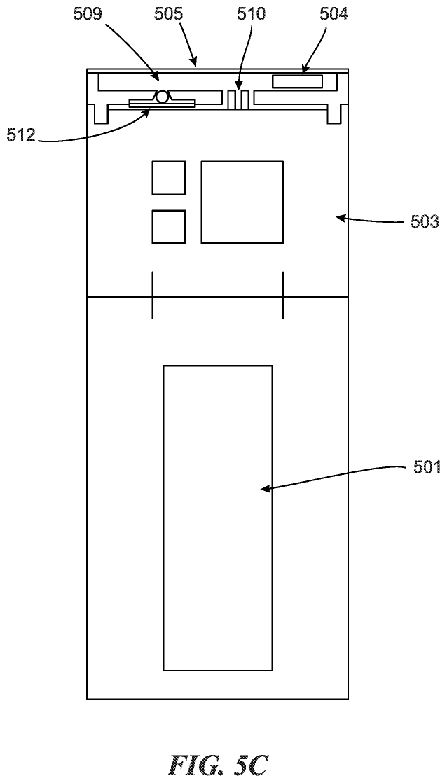

In contrast to integrating the pressure sensor into the deformable lid as shown in the embodiment of FIG. 2, the embodiment of FIGS. 5A-5C provides side and top-down views, respectively, of an embodiment of an AIS 500 that includes a pressure sensor 504 inside a hermetic cavity 509. In the illustrated embodiment, the hermetic cavity 509 can be filled with an incompressible medium such as oil (e.g., silicon oil). The hermetic cavity 509 can include a deformable lid 505, hermetic feedthroughs 514, and a ball and lid 516 configured for filling/draining the hermetic cavity 509 with the medium. By placing the sensor in the hermetic cavity, deformation of the lid 505 can transmit pressure waveforms from inside the patient's heart to the sensor via the medium in the cavity.

In FIGS. 5A-5C, the hermetic feedthroughs 514 can include a plurality of feedthrough pins, such as three pins. The feedthrough connections between the sensor and the electronics can utilize a bus such as I2C, however it should be understood that other buses may be used. Three pins are required for an I2C bus (one pin for VDD, one pin for SDA, and one pin for SCL) assuming the case is the voltage supply source (VSS). The I2C pressure sensor located inside the cavity and bonded to the three feedthrough pins and VSS (not shown). In some embodiments, the deformable lid 505 can comprise a metal such as titanium, MP35N, stainless steel, platinum iridium, or other known biocompatible metals or materials. The lid can be welded (laser, resistance, etc.) to the walls of the hermetic cavity. As described above, the hermetic cavity can be sealed with a biocompatible medium such as silicone oil, and the ball and lid 516 can be pressed, fixed, and/or welded into position to seal the cavity. As shown in FIG. 5C, the hermetic cavity can form the header of the AIS. As shown, the electronics compartment 503 can be attached to the hermetic cavity and electrically coupled to the sensor via hermetic feedthroughs, and the cell 501 can be attached to the electronics compartment and electronically coupled with separate hermetic feedthroughs. The electronics compartment sits in a cylinder in between the hermetic cavity/header and the cell.

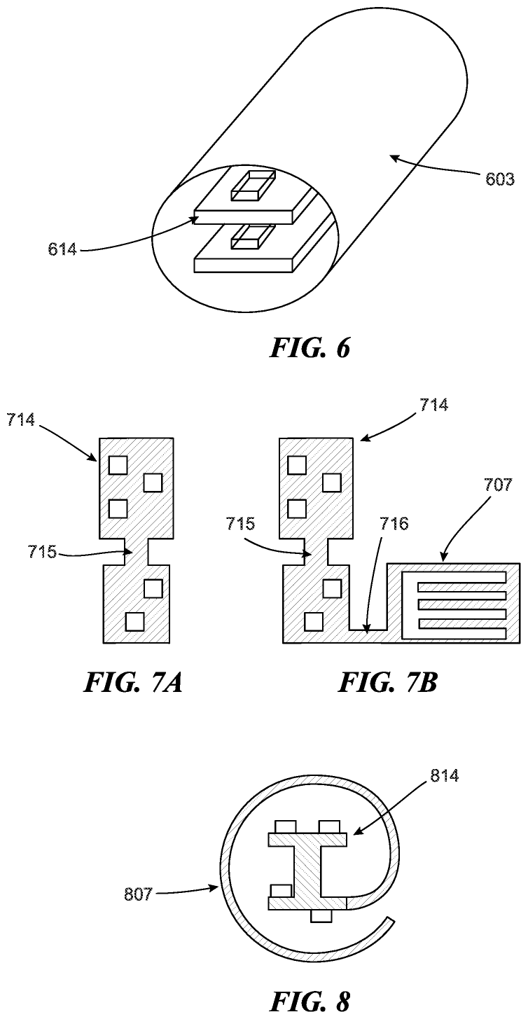

Additional embodiments of the antenna are also provided. In one embodiment the antenna can be wrapped or positioned around electronics within the electronics compartment. Referring to FIG. 6, an electronics compartment 603 of an AIS is shown, with electronics 614 positioned within the electronics compartment. The electronics 614 can comprise any of the electronics described herein, such as a real-time-clock RTC, a microprocessor, a transceiver, a capacitive-to-digital converter or analog-to-digital-converter, etc. In the illustrated embodiment, the electronics can comprise one or more electronic components and/or integrated circuits on a substrate.

FIG. 7A shows one example of electronics 714 that can comprise one or more sections of electronic components and integrated circuits, disposed on sections of substrate connected via a substrate connector 715. In some embodiments, the substrate connector 715 can facilitate bending or folding the electronics to allow for smaller packaging and filling the electronics compartment (as shown in FIG. 6).

In the embodiment of FIG. 7B, the antenna 707 can comprise an extension of the electronics 714. As shown in FIG. 7B, the electronics 714 can comprise one or more sections of electronic components and integrated circuits disposed on sections of the substrate (e.g., flex substrate) and connected via the substrate connector 715. Additionally, in FIG. 7B, the antenna can be an extension of the electronics and be connected to the substrate of the electronics with substrate connector 716. It should be noted that the substrate connectors 715 and 716 can simply be extensions or pieces of the substrate that holds the electronics.

FIG. 8 is a cross-sectional drawing that shows how the electronics and antenna of FIG. 7B could be folded or bent so as to be packaged within the electronics compartment. In this example, the antenna can be wrapped around the electronics and placed inside the cylinder of the electronics compartment. In FIG. 8, the one or more sections of electronics 814 are shown in the center and the antenna 807 is shown wrapped around the electronics 814. This entire assembly can then be mounted or disposed within the electronics compartment (not shown). This solution avoids having to interconnect the substrate from within the electronics compartment to the compartment itself when the antenna is disposed outside the electronics compartment or integrated into the housing/compartment.

The embodiment of FIG. 8 allows the antenna to remain inside the hermetic enclosure of the electronic compartment. In this configuration, the antenna would remain in a hermetic environment. Electric field or magnetic field antenna designs could be realized. Structures could meander, even patch antenna designs could be realized where a ground plane helps to act as a shield to the electronics.

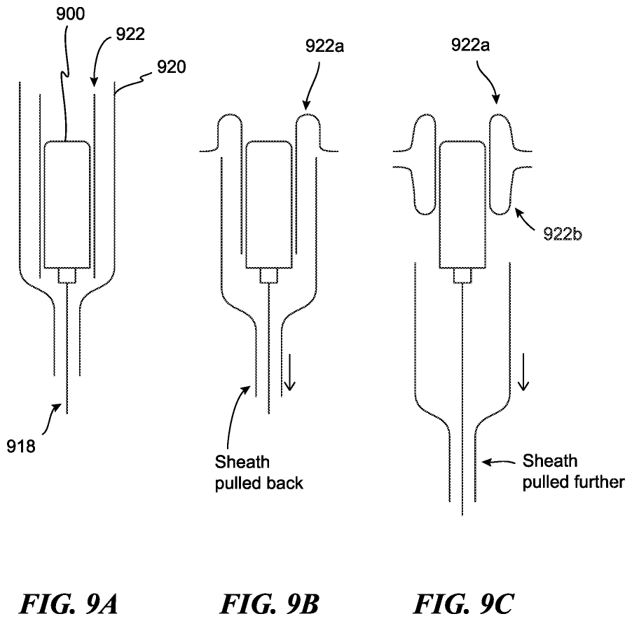

FIGS. 9A-9C illustrate an embodiment for fixation and delivery of an AIS 900. In this embodiment, the AIS can include one or more fixation element(s) 922 that can comprise a front fixation element 922a and a rear fixation element 922b. In this example, the AIS can be loaded onto a steerable catheter and connected with a tether 918. A protective sheath 920 can be advanced or loaded over the AIS to cover the AIS and keep the fixation element(s) 922 constrained during delivery. FIG. 9A shows the sheath fully protecting the AIS and constraining the fixation element 922. In this configuration the device is advanced to penetrate the transeptal atrial wall. In some examples, the sheath and fixation elements are radiopaque. Once the transeptal wall is penetrated, the sheath can be partially retracted to deploy a front fixation element 922a as shown in FIG. 9B. The catheter can then be slightly retracted until the front fixation elements 922a are seen showing resistance against the septal wall. When this occurs, the sheath 920 can be further retracted to expose the rear fixation elements 922b, as shown in FIG. 9C. The catheter is then moved forward and backwards slightly to verify that both forward and rear fixation elements 922a and 922b are engaged with the septal wall. When the device is secure, the tether can be released from the AIS and catheter withdrawn.

As described above, it is an objective to provide an AIS that is self-powered, records pressure information throughout the day (e.g., continuously or semi-continuously), transmits data wirelessly to a relay regularly, has a longevity of at least 5 years, and is housed in a container between 0.5 cc and 2 cc in volume. Below are some of the system requirements for designing such a system that could achieve these goals and meet the longevity requirements.

In a preferred embodiment, the cell of the AIS would be a lithium CFx cylindrical cell. This chemistry is reported to have energy densities upwards of 1000 Wh/l, however these energy densities are not currently achievable in volumes less than 2 cc. A more realistic energy density for CFx is:

$${ED}_{cfx} = 240 \frac{A \cdot hr}{l}$$

The impedance of the cell is also a limiting factor when designing a system, as the current required for wireless telemetry would typically collapse the voltage at the battery terminals.

For example:
R_{cfx}=500Ω is the typical impedance of a small volume CFx cell.
Iₜₓ=16 mA is the typical transmit current for MICS telemetry at 403 MHz.
V_{cfx_internal}=3 V is Thevenins equivalent of the internal voltage of the CFx cell.
V_{cfx_terminal}=V_{cfx_internal}−Iₜₓ·R_{cfx}=−5 V. This shows that the cell can clearly not support continuous transmit.

The following analysis addresses these limitations to describe an AIS that achieves the desired longevity in a volume of less than 2 cc. A few assumptions can be made for the system to get an idea of the design objectives:
I_{q}=1.5 μA is a reasonable quiescent current of the device.
V_{cell}=0.4 cm³ is a reasonable volume committed to the cell.
Q_{cell}=ED_{cfx}·Vcell=96 mA·hr provides the usable cell capacity.

$${T}_{life} = \frac{{Q}_{cell}}{{I}_{q}} = 7.3 yr$$

provides the longevity of the cell.

Now, assume the AIS can measure blood pressure continuously at very low current and transmit this data without interruption.

n=8 bits is the number of bits per blood pressure sample.

Tₛ=100 Hz is a common sampling rate required for blood pressure.

γ=4.6 is a compression ratio using double turning point.

$${N}_{day} = \frac{n \cdot 86400 s \cdot {T}_{S}}{γ}$$

N_{day}=(1.878·10⁶) bytes is the number of bytes to be sent out daily by an AIS continuously measuring blood pressure.

In one preferred embodiment, the AIS would use a MICs band transceiver, such as the CC1101 transceiver by Texas Instruments.

CC1101:

$$data\_rate = \frac{500000 \cdot bit}{s}$$

is the fastest data rate.

$${T}_{tx} = \frac{{N}_{day}}{data\_rate} = 30.1 s$$

is the time required to send information.

I_{lp_tx}=16 mA is the transmit low-power current consumption.

I_{lp_rx}=16.5 mA is the receive low-power current consumption.

Q_{day}=Tₜₓ·I_{lp_tx}=0.134 mA. hr is the charge required only to send continuous blood pressure every day.

$${T}_{life} = \frac{{Q}_{cell}}{{Q}_{day}} \cdot day = 1.97 yr$$

is how long continuous blood pressure result could be sent, resulting in poor longevity, even if the cell terminal voltage could support the current drain.

Rather than sending the entire waveform each day, the implant can send daily statistics that can be synthesized from blood pressure measurements taken throughout the day. For example, instead of sending the entire blood pressure waveform data, the AIS can compile hourly measurements of systolic, diastolic and mean pressure, as well as heart rate statistics such as mean heart rate and variability. In the next example, let's transmit small packets of data, where the payload is small enough to prevent the cell terminal voltage from collapsing and the time to the next packet is long enough for the supply holding capacitor (e.g., supply holding capacitor 112 in FIG. 1) to recover.

δ=11·byte is the minimum packet overhead at the max data rate.

nₜₓ=4·byte is sending only 4 bytes of data per packet to keep the holding capacitor small.

$${T}_{packet} = \frac{{n}_{tx} + δ}{data\_rate} = 0.24 ms$$

is the packet transmit times, including overhead.

T_{idle_to_tx}=75.2 us is the idle to transmit time for the CC1101 datasheet.

T_{TX}=T_{idle_to_tx}+T_{packet} is the time the transmitter is on.

Q_{tx_packet}=I_{lp_tx}·T_{TX}=1.401 nA·hr is the charge required for packet transmission.

C_{hold}=47 μF is a reasonably small capacitor to keep leakage down.

Iₗₑₐₖ=200 nA is an estimate of current leakage.

$${V}_{drop} = \frac{{Q}_{x \_ packet}}{{C}_{hold}} = 107.3 mV$$

Therefore, in this example, a 47 μF supply holding capacitor is sufficient to prevent the cell voltage from collapsing approximately 100 mV. Now, only sending 4 bytes per packet may not be sufficient, so let's send at least 8 packets and double it again in case of poor reception by the relay or the overhead needed for encryption.

σₜₓ=8·2 is the daily number of packets to send.

$${Q}_{tx \_ daily} = {Q}_{tx \_ packet} \cdot {σ}_{tx} = 22.4 nA \cdot hr$$

$${T}_{life} = \frac{{Q}_{cell}}{{I}_{q} + \frac{{Q}_{tx \_ daily}}{day} + {I}_{leak}}$$

Now, let's take into account the battery capacity required for receiving an acknowledgement from the relay.

nᵣₓ=4·byte is sending only 4 bytes of data per packet to keep the holding capacitor small.

σn=2 is the assumption that only 1-2 packets are necessary to receive and acknowledgement from the relay.

$${T}_{rx} = \frac{{n}_{rx} + δ}{data\_rate} = 0.24 ms$$

is the receive time for one packet, including overhead.

T_{idle_to_rx}=75.1μs

T_{RX}=T_{idle_to_rx}+T_{packet} is the time the transmitter is on.

Q_{rx_packet}=I_{lp_rx}·T_{RX} is the charge required for packet reception.

Q_{rx_daily}=Q_{rx_packet}. On is the daily charge due to packet reception.

$${T}_{life} = \frac{{Q}_{cell}}{{I}_{q} + \frac{{Q}_{tx \_ daily}}{day} + \frac{{Q}_{rx \_ daily}}{day} + {I}_{leak}} = 6.4 yr$$

is the longevity for both TX and RX.

C_{hold}·R_{cfx}·5=117.5 ms tells us to wait at least 120 ms for the holding capacitor to recover to 99.3% of the final value.

The conclusions above tells us that it is possible to transmit data wirelessly from an AIS by using a small volume CFx cell by breaking it up into small packets and spreading the packets over time to prevent the collapse of the cell terminal voltage while maintaining a reasonably low quiescent current drain. While it may be possible to design a custom ASIC to measure blood pressure with a reasonable sample rate and let the ASIC calculate systolic, diastolic and mean parameters on a cycle-by-cycle basis to achieve a system goal of 1.5 microamps or less, it's currently not possible to do this with existing off-the-shelf components.

However, by sampling blood pressure, say N seconds every X minutes it's possible that an off-the-shelf solution can be found and still achieve the quiescent current drain specification given reasonable values for N and X.

Let's look at doing this with an off-the-shelf solution. First the AIS needs a capacitive-to-digital converter to process the signal from the pressure sensor. The ESS113 by ES Systems reports a maximum consumption of 350 microamps for sampling rates up to 195 Hz. Let's calculate the current for sampling at 100 Hz.

I_{ESS113_OFF}=0.6 μA is the reported standby current drain.

I_{ESS113_MAX}=350 μA is the maximum current at 195 Hz sampling.

$${I}_{ESS 113\_ 100 Hz} := {I}_{ESS 113\_ MAX} \cdot \frac{100 Hz}{195 Hz}$$

I_{ESS113_100Hz}=179.5 μA is the current drain at 100 Hz.

And now, let's assume that we're running an off-the-shelf microprocessor at 3 V.

I_{MICRO_ON}=300 μA is the estimated micro current drain when active.

I_{MICRO_OFF}=0.2 μA is the estimated standby current drain.

Adding up the standby current drains yields:

Iₗₑₐₖ+I_{ESS113_OFF}+I_{MICRO_OFF}=1 μA so we only have 500 nA available for the blood pressure measurement.

Let's see how we can adjust the duty cycle of the pressure sensor data acquisition to accommodate 500 nA.

N=3.75 sec is the time to sample the pressure sensor waveform.

X=1 hr is the period selected to start sampling.

$$D = \frac{N}{X} = 0.001$$

is the duty cycle.

I_{ACTIVE}=I_{MICRxO_ON}+IESS113_100 Hz is the current drain for active components.

I_{ACTIVE}·D=499.5 nA is the average current drain when duty cycled. Therefore, a pressure waveform sampling rate (e.g., sampling N seconds every X minutes) can be chosen to maintain an average current drain (e.g. 500 nA or less), within the system constraints defined above.

In the preferred embodiment the AIS would be constructed with a cell and hermetic compartment for the electronics. This compartment would be typically be sealed at atmospheric pressure. The compartment can include a lid that incorporates a MEMs capacitive sensor, whereby the pressure of the blood exerts a force on the lid to deform it, causing a change in capacitance. This pressure signal is not a true gage pressure measurement as required for blood pressure because the reference pressure is fixed. To compensate, the relay contains an atmospheric pressure sensor that records calibration atmospheric pressure data throughout the day. The pressure data from the implantable sensor and the calibration atmospheric data are combined either in the relay or at the data center to achieve the designed gage measurement of blood pressure.

The key to developing a long-term implantable pressure sensor is to make sure the pressure sensing area is sufficiently large enough to not be affected by the eventual tissue encapsulation that will form. Off-the-shelf sensors typically have a small surface area and use materials that lack biocompatibility. The embodiment described in FIGS. 5A-5C demonstrates how to translate the small surface area provided typical pressure sensors into a larger pressure sensing area that will be less sensitive to the effects of tissue encapsulation. As for additional details pertinent to the present invention, materials and

manufacturing techniques may be employed as within the level of those with skill in the relevant art. The same may hold true with respect to method-based aspects of the invention in terms of additional acts commonly or logically employed. Also, it is contemplated that any optional feature of the inventive variations described may be set forth and claimed independently, or in combination with any one or more of the features described herein. Likewise, reference to a singular item, includes the possibility that there are plural of the same items present. More specifically, as used herein and in the appended claims, the singular forms “a,” “and,” “said,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs. The breadth of the present invention is not to be limited by the subject specification, but rather only by the plain meaning of the claim terms employed.

1. An active implantable pressure sensor, comprising:
a housing adapted to be implanted within a human heart;
a cell disposed in the housing;
a pressure sensor disposed in the housing and configured to measure pressure information outside of the housing;
a transmitter disposed on or in the housing and configured to transmit the pressure information;
a supply holding capacitor electrically connected to the cell, the supply holding capacitor being configured to provide current to the transmitter during transmission; and
a microprocessor configured to control transmission of the pressure information by the transmitter, wherein the microprocessor is configured to intermittently turn on the transmitter for a transmit-time sufficient to avoid a supply voltage of the active implantable pressure sensor from collapsing and turn off the transmitter for an off-time sufficient to allow the cell to recharge the supply holding capacitor above a recharge threshold.

2. The sensor of claim 1, wherein the housing comprises a separate hermetically sealed cavity for housing the pressure sensor.

3. The sensor of claim 2, wherein the separate hermetically sealed cavity is oil filled.

4. The sensor of claim 2, wherein the separate hermetically sealed cavity comprises a deformable lid.

5. The sensor of claim 3, wherein the separate hermetically sealed cavity further comprises an oil fill port that is sealed with a ball and lid.

6. The sensor of claim 1, wherein the pressure sensor comprises a MEMs capacitive sensor.

7. The sensor of claim 1, wherein the housing comprises a cell compartment for housing the cell, an electronics compartment for housing the transmitter and microprocessor, and a hermetically sealed cavity for housing the pressure sensor.

8. The sensor of claim 7, wherein the electronics compartment comprises a volume of between 0.25 and 0.75 cc.

9. The sensor of claim 1, further comprising an antenna operatively coupled to the transmitter.

10. The sensor of claim 9, wherein the antenna is integrated into the housing.

11. The sensor of claim 9, wherein the antenna is hermetically sealed within the housing.

12. The sensor of claim 1, wherein the transmitter and microprocessor are disposed on a substrate.

13. The sensor of claim 12, wherein the substrate comprises a flex substrate, wherein the flex substrate is folded or bent to fit in the housing.

14. The sensor of claim 13, wherein further comprising an antenna disposed on the flex substrate.

15. The sensor of claim 14, wherein the antenna is folded or wrapped around at least a portion of the transmitter or microprocessor.

16. The sensor of claim 12, wherein the substrate comprises a first section and a second section attached with a connecting portion of substrate, wherein the substrate is configured to bend or fold on the connecting portion, wherein the transmitter and microprocessor are disposed on the first section and second section.

17. The sensor of claim 16, wherein the substrate further comprises a third section attached to the second section with a second connecting portion of substrate, wherein the third section of substrate includes an antenna.

18. The sensor of claim 1, wherein the sensor is configured to have an operating lifetime of at least 5 years.

19. The sensor of claim 18, wherein the operating lifetime of 5 years is based on a housing volume between 0.5 cc and 2.0 cc and a cell volume of up to 0.75 cc.

20. The sensor of claim 1, wherein the housing has a volume of between 0.5 cc and 2.0 cc.

21. The sensor of claim 1, wherein the microprocessor is configured to synthesize and compress the pressure information before transmission.

22. The sensor of claim 1, further comprising a giant magnetoresistance (GMR) sensor.

23.-98. (canceled)
